(19) United States
(12) Patent Application Publication
(10) Pub. No.: US 2020/0128887 A1
(43) Pub. Date: Apr. 30, 2020

(54) PORTABLE COOLING DEVICE FOR HUMANS AND/OR ANIMALS

(71) Applicant: Heinrich Raimund LABUS, Monheim (DE)

(72) Inventors: Heinrich Raimund LABUS, Monheim (DE); Andreas LABUS, Burscheid (DE)

(73) Assignee: Heinrich Raimund LABUS, Monheim (DE)

(21) Appl. No.: 16/473,325

(22) PCT Filed: Feb. 13, 2018
(86) PCT No.: PCT/EP2018/053517

§ 371 Date: Jun. 25, 2019

(30) Foreign Application Priority Data
Feb. 23, 2017 (DE) 10 2017 103 731.4

(51) Int. Cl. A41D 13/005; A61F 7/00; A01K 13/00; A62B 17/00

(52) U.S. Cl. CPC A41D 13/0056; A61F 7/00; A01K 13/006; A61F 2007/0234; A61F 2007/0062; A61F 2007/0065; A61F 2007/0068; A62B 17/005

(57) ABSTRACT

The invention relates to a portable cooling device for humans and/or animals, in particular a vest, a jacket, a cape, a coat or a blanket, characterized in that the portable cooling device (10) comprises at least two water atomizers, which are connected thereto, together with the reservoirs (2.2, 5) and at least two fans (2.1, 6), in that the portable cooling device (10) has a fixing device so that, when in use, a free ventilation and cooling region (11, 12) is formed between the portable cooling device (10) and the surface to be cooled (13, 14) of the human and/or animal, and in that the fans (2.1, 6) and the water atomizers together with the reservoirs (2.2, 5) for ventilating and cooling said ventilation and cooling region (11, 12) are arranged next to one another.

The invention relates to a portable cooling device for humans and/or animals for attaching to the upper body and head region, in the form of a cape, a jacket or vest or blanket for protection in the event of a high ambient temperature.

The invention relates in particular to a cooling device for the upper body of a human, and correspondingly modified also for the front of the body of pets, in particular for dogs and horses, for protection at high ambient temperature.

In activities of humans and animals in an excessively hot and usually also sunny environment the body protects itself against overheating by sweating, in order to lower the raised body temperature again to a normal level and to stabilize it. However, over a relatively long time period such a state of strain on the body is harmful to health or even life-threatening, because in addition to dangerous overheating in this case a relatively large amount of fluid and vital minerals in addition to other substances are permanently excreted. Among the pets in question, in particular dogs are susceptible to heat, because they can only dissipate the excess heat slightly by means of sweating. In dogs the majority of the heat dissipation takes place predominantly by panting, which is not particularly effective because of the small evaporation surface.

### PRIOR ART

Several possible solutions for the individual protection of the human or animal body against overheating are known from the patent literature. The devices described there for humans are generally designed in the form of a vest. One of the first cooling devices is based on continuous ventilation of the space between the upper body and a jacket with built-in fans (EP 490 347 A1). Another cooling concept is based on a vest with porous inserts and fans. The inserts here serve as spacers and at the same time as an air passage for ventilation of the upper body. If required, cold or heated air could be used alternately (US 2007/0118956).

A device for air conditioning the human body is also known in the form of a coat with attached head covering, equipped with a complex internal installation. The ventilation takes place using outside air with the aid of a fan, heating element and battery, which are accommodated in an external box.

The heating element can be switched on as required for heating the air that is used (U.S. Pat. No. 3,468,299 1969).

Another principle for cooling the upper body is based on air pumped in circulation in channels provided therefor in the vest. The air circulating therein, in contact with the upper body, continuously extracts some of the excess heat as well as sweaty moisture and then flows into an air conditioning box, where it is returned to the circulation after drying and cooling (WO 2014/031 128 A1).

Another patent protects an air-conditioned safety vest with additional protective equipment for special applications. The air conditioning of the user takes place by ventilation with the aid of a plurality of fans with ambient air, which can be heated electrically as required. The electric battery is recharged by the solar panel attached in the back region of the vest (US 2012/0036623 A1).

A fundamentally different cooling principle, using exclusively water, differs substantially from the modes of operation described above. Accordingly, however, the cooling device in the form of a vest consists of a woven fabric with additions which have reversible water-storing properties. The water previously introduced periodically into the vest before it is used for cooling then evaporates gradually during the cooling and thereby extracts the excess heat from the region to be cooled (DE 10 2011 016 482 A1). The cooling vests functioning according to this principle are commercially available for specific occupational activities or for motorcyclists or for animals, such as dogs or horses in the form of cooling jackets or cooling blankets.

The device described in the patent US 2013/0319031 A1 also operates according to the cooling principle of upper body ventilation. For effective air distribution two flat chambers are provided which are arranged one above the other. The cooling device is primarily intended for use together with a bullet-proof vest.

The ventilation takes place with the aid of fans and battery.

Another portable device for humans, designed for cooling of the back, is based on the use of air and atomized water as coolant (U.S. Pat. No. 6,543,247). It is composed of an electrically powered fan and a water atomizer with conventional air/water nozzle. These components are accommodated in a housing with a widened outlet of the Venturi type for the cooling medium flowing out. The water is atomized directly in the airflow of the fan, guided under the clothing along the back and continuously cooled. The above cooling principle has also been implemented already in another cooling device as a handheld device and marketed under the brand name “Arctic Blast.”

In cooling vests with forced air circulation in the region between the upper body and the facing side of the cooling vest, the effect is dependent not only on the external temperature and the relative air humidity, but also on the physical features of the specific user. The excess amount of heat to be drawn off actually varies from person to person to a great extent, depending upon body weight, size, age, and physiological features, and naturally also depends on the state of health and the activity currently being undertaken by the person to be cooled. In addition to the air speed on the upper body during the cooling operation, the quantity of sweat which forms exerts a significant influence on the effectiveness of cooling, in particular at outside air temperatures above for instance 35° C.

In the known devices according to the principle of cooling with ambient air there is unfortunately no possibility of compensating for these factors.

The cooling vests according to the principle of water storage in the woven fabric having subsequent evaporation have many disadvantages. Since the evaporation of water and thus also the resulting lowering of the temperature occur directly on the surface of the wet woven fabric, the cooling of the wet vest fabric preferably takes place. Therefore, the desired cooling of the human body proceeds indirectly and with a time lag. Because of the lack of ventilation here and the resulting gradual increase in the moisture content in the space between the cooling fabric and the body to be cooled, the cooling operation is additionally hampered. Furthermore, the sweat which forms can be evaporated and dissipated only with difficulty or hardly at all. In addition, the cooling is not controllable.

Another disadvantage is the high initial weight of the vest due to the relatively large quantity of water stored and the very high and ineffective water consumption. The use of such a cooling technique in a dry area is naturally restricted because of the water shortage usually prevailing there. One of the serious disadvantages of the known cooling devices with an air/water mixture as cooling medium is the relatively large drops of water which are specific for the conventional air/water nozzles provided there. Moreover, the size of the drops is spread over a wide range of for instance 100 to 20 μm (source: Ecologic Technologies, Inc., 2017). In addition, the introduction of the atomized water into the airflow promotes the combination of smaller drops into larger drops. Therefore, after reaching a critical quantity and thus a critical mass, these drops are precipitated from the air stream and will only play an insignificant part or no part at all in the further cooling operation. In this case most of the remaining drops are also on average larger, and as a result their total surface by comparison with the initial state is substantially smaller and the effectiveness of the cooling is accordingly poorer. The precipitated drops of water collect in the device and can often lead to hygiene problems. A further disadvantage is the uncontrollable quantity of air, and for this reason the cooling efficiency cannot be adapted optimally. At an above-average high external temperature the problem exists that the heat contained in the excessively strong air inflow would level the cooling effect of the atomized water or would even contribute to the additional heating of the upper body. As a result of the unsatisfactorily cooling effect and to some extent also because of the complexity of the devices known from the prior art, these are suitable in particular for activities in the sports and leisure sector.

### OBJECT OF THE INVENTION AND SOLUTION

The object of the present invention is to provide portable cooling devices for humans and animals, in particular for dogs and horses, designed above all for an above-average hot environment. The cooling devices should be compact and also simple in construction and application. In a sunny area it should be possible to use them largely autonomously in terms of energy. They should fill the existing gap in suitable cooling devices for occupational and other activities of humans and animals in hot areas.

In a portable cooling device for humans and/or animals of the type referred to above, according to the invention this object is achieved in that the portable cooling device has a rucksack-like receptacle or a cooling device in a simplified version, consisting of correspondingly modified belts with power connection and at the same time comprises, as a substrate for the correspondingly small cooling components for humans or a vest or blanket in the case of animals, at least in each case two ultrasound water atomizers connected thereto together with reservoir, and at least two fans, so that, in use, a free ventilation and cooling region is formed between the portable cooling device and the surface of the human and/or animal to be cooled, and the fan and the water atomizer are arranged adjacent to one another for ventilating and cooling this ventilation and cooling region.

The following advantages are achieved inter alia:

The cooling devices for humans or animals according to the invention are based on the continuous extraction of the excess body heat by continuous atomization and evaporation of water as well as discharge of the water vapor by adjustable ventilation of the body area to be cooled. The cooling of the intensively heat-releasing regions, such as the upper body in the case of humans and the front of the body in the case of animals, is particularly effective. The cooling effect according to the invention is primarily achieved by evaporation of the water which is continuously very finely atomized with the aid of ultrasonic waves in the immediate proximity of the body area to be cooled and of the head. Due to the cooling as a result of the evaporation of the finely atomized water directly in the region to be cooled, and because of the very minimal ventilation of this region, the cooling devices for humans and animals according to the invention are also still usable as a rule at external temperatures above 35° C. The very high temperature and moisture content in the region to be cooled naturally facilitate colonization and growth of microorganisms. In order to prevent the undesirable propagation thereof, according to the invention for the atomizer a salt-free water with low addition of hydrogen peroxide is used as biocide, which in these circumstances raises no concerns for humans and animals.

In the cooling device according to the invention the ambient air is required for ventilation of the cooling region and for discharging the water vapor formed and only secondarily as a coolant. Therefore, above an external temperature of approximately 35° C. the air flow merely has to ensure sufficient ventilation of the region to be cooled and can remain limited thereto. The cooling effect according to the invention is in general much more effective than with air alone and is also still possible at external temperatures above 37° C. As a result of the better cooling effect of the device according to the invention, the troublesome excessively sweating is largely suppressed.

The ambient air used for cooling and the atomized water are preferably controllable in terms of quantity, in order to adapt the cooling power to the currently prevailing external temperature and to configure it optimally. In the cooling devices according to the invention the quantity control can be carried out in a simple manner, by changing the electrical voltage at the fans and/or the water atomizers with the aid of voltage controllers in continuous operation or in selectable time intervals.

For the required electrical energy for ventilation and for atomization of water, the device according to the invention is equipped with a battery. This can be charged periodically as required from the electricity network and during operation in a sunny area it can be recharged continuously by a solar panel. In this way the cooling devices according to the invention can even be self-powered. In order to safeguard an optimal current yield, the solar panel is oriented horizontally or according to the position of the sun overhead. In addition, or alternatively, if required, smaller solar panels can also be positioned on the shoulders.

According to the invention an outer cover for the upper body and a part of the device, in the form of a cape or a jacket, for example, serves for protection against heat from outside. The main feature of this thermal shield according to the invention is the metal-coated or other type of heat-reflecting outer surface thereof and a thermal insulating layer on the underside of the cover, manufactured from woven fabric and/or film, and an additional woven fabric lying above it which is partially light-permeable, matt and as a result light-scattering, and/or a corresponding film. Otherwise the shield can be configured in any way and adapted to fashion trends or to local clothing habits and can be changed as required. The externally directed woven fabric or the corresponding film principally has a masking function, because it covers the metal-coated layer and as a result practically no disruptive light reflection is perceptible. The purpose of the metal-coated surface of the shield according to the invention is to reflect as much as possible of the incident thermal radiation and as a result to improve the effectiveness of the cooling device.

The cooling device for humans according to the invention is based on the cooling of the upper body and of the head, because the excess heat is given off particularly intensively in these regions. It consists of a backpack-like receptacle with carrying straps and an attached, collapsible framework as support for the solar panel and for a sunshade positioned in the top region with a fan and water atomizer fixed thereon.

The wall of the receptacle facing the back has a fleece-like or felt-like, sufficiently coarse-structured surface in order to ensure optimal conditions for the evaporation in the region to be cooled between the back and this container wall and thus to ensure the highest possible efficiency of the cooling effect of the atomized water, and furthermore to ensure a sufficient intermediate space for effective ventilation. The structured surface simultaneously functions as a temporary absorber for the radiant heat from the upper body and furthermore also as a reversible, equalizing intermediate store for a small part of the atomized water, which evaporates immediately under the effect of the absorbent heat and is continuously removed from this region by ventilation. Studies showed that the heat discharge by radiation from the human body plays a significant role. For example, it was ascertained that in a resting human at 20° C. the heat discharge takes place through: heat radiation 46%, convection 33%, sweating 19% and respiration 2% (Prof Dr.-In. E. Specht, Uni. Magdeburg, “Der Mensch als wärmetechnisches System” [“The human as thermal system”], 2005).

The cooling module for the back is located in the lower region of the receptacle. It consists of one or more fans, one or more ultrasonic water atomizers, an electric storage battery, a voltage controller and/or a voltage pulse controller with adjustable duration. The fans and ultrasonic water atomizers are arranged adjacent to one another and combined with the other components into a module which is removable from the receptacle.

Alternatively, the ultrasonic water atomizers, fans, voltage controller and battery can be individually attached directly to particularly suitable points on a belt modified according to the invention and, as a modified and compact version of the previously described autonomous cooling device, it can be used in this convenient embodiment at any time and anywhere as required. For this purpose, according to the invention the modified belt is used with two parallel rows of magnetized rivets, the rows being connected in each case by an electrical conductor both to one another and also to the battery attached to the belt. In addition to the possibility of electrical connection of the ultrasonic water atomizers and fans to the battery, the belt also serves at the same time as a substrate for these cooling components. For the purpose of simple fixing with simultaneous electrical connection of the fans and water atomizers to the battery at any point on the belt, the rivets in the row are in each case connected to one another and to one of the battery poles. The fans and water atomizers are in each case accommodated in a housing having openings for air. Neodymium magnets which are electrically insulated from one another, and which are in each case positioned on the belt congruently with one of the rivets in the upper or lower row, are fixed on the housing surface facing the belt. On the other hand, these magnets are electrically connected to the fan or water atomizer. The magnets not only ensure a correct and reliable electrical connection to the battery, but simultaneously serve as a simple and quickly operated means for fixing to the belt, placed at points at which the user perceives the cooling effect as optimum in given conditions. The heat-insulating jacket described above serves for protection against heat from outside. This very simplified, very small and in this connection lightweight cooling device according to the invention is nevertheless sufficiently powerful and hardly disruptive in use. Therefore, it can be used anywhere without restriction.

A thermal protection cover in the form of a cape or a jacket of the two-part construction already described above, specifically a light-scattering coating and a heat-insulating layer composite below it which reflects light or thermal radiation, serves for thermal shielding of the upper body and of a part of the cooling device. The framework together with the installation in the top region as well as the thermal protection cover can be collapsed again after use and stowed in the receptacle. The cooling device then forms a convenient, compact unit and can be transported or stored as a backpack.

Advantageous embodiments of the invention are set forth in the sub-claims.

Thus, it is provided
that the portable cooling device (10) for a human has a form such that, during use, the ventilation and cooling region is formed between the receptacle and the back of the body,
that the receptacle wall facing the upper body is structured, preferably in the form of protrusions, such that, during use, the ventilation and cooling region is formed between the receptacle and the back of the body,
that the cooling device for animals is constructed in the form of a heat-insulating vest or blanket and the inner faces thereof are structured so that the cooling of the animal body under it is possible,
that the cooling is achieved practically only due to the evaporation of atomized water with the aid of ultrasonic waves directly in the region to be cooled, and the ventilation by means of ambient air serves merely for discharge of the water vapor which forms,
that the water used for the cooling, which is very finely atomized by ultrasound, and the ambient air used for discharging the water vapor which forms through ventilation are controllable in terms of quantity, in order to adapt the cooling power of the currently prevailing external temperature and to configure it optimally, in particular by changing the electrical voltage on the fans and/or the ultrasonic water atomizers with the aid of voltage controllers,
that for the required electrical energy for ventilation and for atomization of water the device is equipped with a storage battery, which in particular can be charged periodically as required from the electricity network and which during operation in a sunny area can be recharged continuously by a solar panel.
that in order to safeguard an optimal current yield the solar panel is oriented horizontally and/or according to the position of the sun overhead,
that an outer cover for the upper body and a part of the device, in the form of a cape or a jacket, for example, is provided for protection against heat from outside,
that the cover has a three-layered construction of heat-insulating composite layers which scatter light and reflect thermal radiation.

### EXEMPLARY EMBODIMENTS

Several embodiments of the invention are explained in greater detail below with reference to the drawings. In all the drawings the same reference signs have the same significance and therefore are optionally explained only once.

In the drawings:

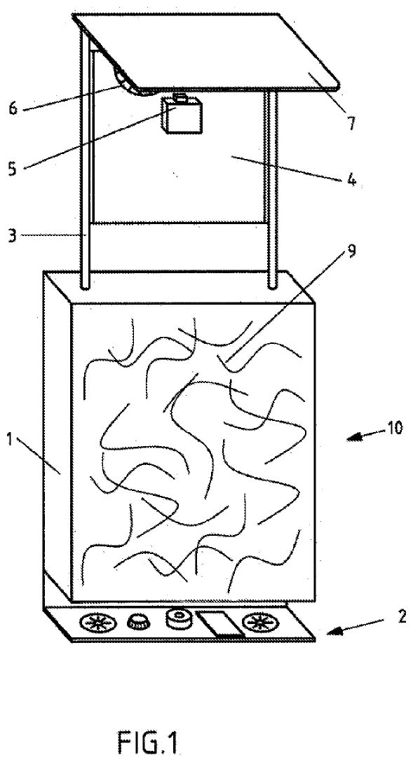

FIG. 1 shows a perspective representation of the portable cooling device according to the invention,

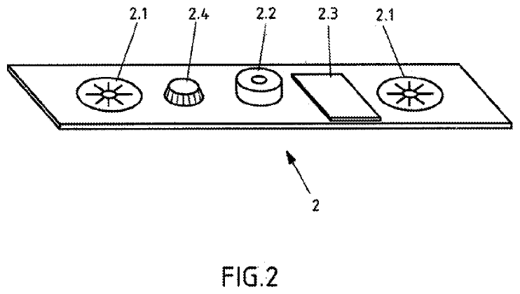

FIG. 2 shows the cooling module 2 of FIG. 1, and

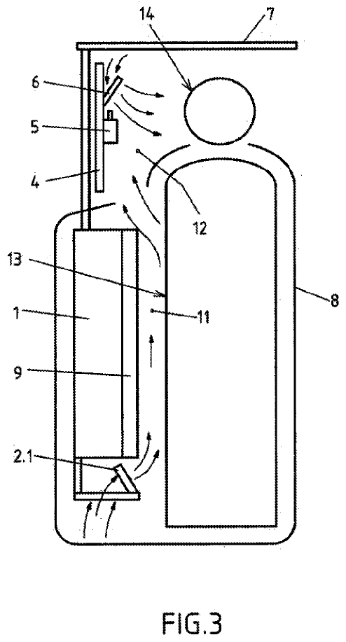

FIG. 3 shows a side view of the portable cooling device according to the invention as shown in FIG. 1.

### GENERAL

The devices according to the invention serve for continuous cooling of the body of a human or animal to a more tolerable level during a stay in a particularly warm environment.

### Example 1 of the Cooling Device for Humans According to the Invention (FIGS. 1 to 3)

The device for humans according to the invention (FIGS. 1 to 3) consists of a receptacle 1 with carrying straps, a removable module 2 with two fans 2.1, an ultrasonic water atomizer together with reservoir 2.2, an electrical battery 2.3 and a voltage controller 2.4.

Furthermore, the device consists of an extendable framework 3 with a sunshade 4 mounted thereon with additional cooling in the top region, consisting of an ultrasonic water atomizer together with reservoir 5 and a fan 6, and ends with a solar panel 7 which is positioned above the head horizontally at the upper end of the framework 3.

A very important component of the cooling device according to the invention is the thermal protection cover 8 in the form of a cape or a jacket which also partially covers the receptacle 1.

The thermal protection cover according to the invention is characterized by a three-layered construction, specifically light-scattering, heat-reflecting and heat-insulating layers.

The cooling principle of the devices for humans and animals according to the invention is based on particularly effective evaporation of continuously finely atomized water to mist and as a result extraction of the heat, both on the body and also in the top region and subsequent discharge of the resulting water vapor by the ventilation, principally limited thereto, of the regions to be cooled with outside air. The inflow of water mist as well as the intensity of ventilation can be adjusted as required by means of the voltage controller 2.4. The moisture-enriched air continuously leaves the cooling zone in the upper region of the cover 8.

Use of the Cooling Device

Before the cooling device is used the electric storage battery 2.3 is charged, the water container on the atomizer 2.2 is filled, and the sunshade 4 with the ultrasonic water atomizer 5 and the fan 6 in the top region and the solar panel 7 above the head are positioned and fixed.

After the attachment to the back of the receptacle 1 with the other components of the device accommodated therein and the fitting of the thermal protection cover 8 over the upper body as well as the receptacle 1 and the switching on of the current supply on the battery 2.3, in the back region the cooling begins as a result of the evaporation of the continuously fed, finely atomized water. Ambient air flows through the space 11 between the container wall with structured surface 9 facing the back and the back 13, in order to absorb the moisture which forms and subsequently to escape in the neck and back region of the thermal protection cover 8.

The sunshade 4 with the water atomizer 5 integrated therein and the fan 6 ensure the cooling in the top region. As a result the positioning of the solar panel 7 horizontally above the head guarantees an optimal current yield and ensures a continuous recharging of the electric storage battery 2.3 independently of the direction of movement of the human wearing the cooling device. After use, the framework 3 and the solar panel 7 are collapsed and together with the thermal protection cover 8 are accommodated in the associated receptacle 1.

### Example 2 of the Cooling Device According to the Invention for a Dog

The vest for dogs can be made of a heat-insulating material, for example a stable, resilient plastic with microscopically small gas inclusions and an aluminum coating on the outer face. The outer surface of the vest is provided with a flexible solar panel. The inside of the vest is correspondingly structured in order to ensure optimal cooling and flow conditions.

The cooling module, consisting of fan and water atomizer, battery and voltage controller, is attached to the front or back of the vest and is accessible on the underside of the vest for cooling media. During the cooling operation the water mist produced in the atomizer evaporates between the inner side of the vest and the body surface and the excess heat is continuously extracted thereby from the body part to be cooled. The water vapor which is formed is discharged by ventilation of the region to be cooled to the outside.

The cooling device according to the invention for a horse is analogous to that for a dog, albeit having taking account of the differences in the anatomy and the envisaged animal use. In the case of a riding horse, for example, the cooling device can consist of two segments, wherein the cooling is focused primarily on the chest region of the horse. The second segment serves for attachment of the solar panel and potentially additional cooling modules.

Several important details of the invention are summarized below.

The cooling device comprises water atomizer, fan, receptacle or vest, an electric storage battery, voltage controller, a sunshade with water atomizer and fan attached thereto, a solar panel and a thermal protection cover.

The extraction of heat in the region to be cooled takes place principally by atomization of water, preferably using ultrasonic waves.

The air used for ventilation of the region to be cooled between the body and the receptacle or the vest serves primarily for discharging the resulting water vapor from the cooling zone.

The container wall facing the back or the vest of the device has a surface-enlarging structure, preferably in the form of protrusions.

The framework serves as a substrate for the sunshade with the attached water atomizer and fan and also for horizontal fixing of the solar panel above the head. The framework can be collapsed and stowed in the receptacle.

The heat-insulating cover 8 preferably consists of two parts arranged one above the other. The upper, free part of the cover preferably consists of a light-scattering woven fabric, a film or a composite of both materials. The part of the cover lying below it preferably consists of a woven fabric, a film or a composite of both materials, wherein the upwardly directed surface additionally has a heat-reflecting, preferably metallic coating, and on the rear surface has a heat-insulating coating.

The vest for animals is preferably made of a heat-insulating plastic, preferably with microscopically small gas inclusions, and has a structured underside and a metal-coated or other heat-reflecting outer face.

These devices serve for removing excess body heat regions which give off heat particularly intensively, such as the upper body and the head.

In this case finely atomized water is introduced continuously into the regions to be cooled, evaporates quickly there and thereby intensively and at the same time effectively extracts heat from the immediate environment of the body to be cooled.

In addition, a controlled ventilation of the body regions to be cooled takes place. The purpose of the ventilation is above all to discharge the water vapor which forms. In detail, the embodiment of the device for humans according to the invention consists of a receptacle with attached carrying straps, fans, water atomizers and a framework for fixing a head cooler as well as a solar panel. The effective cooling zone is formed during the use between the receptacle and the back of the body. During the cooling operation the upper body and a part of the device are shielded against the heat from outside by putting on a heat-insulating cape. When collapsed, the cooling device forms a convenient and lightweight unit which can be used at any time.

The cooling device for animals is correspondingly modified. Otherwise it consists of the same or similar components and functions in a manner analogous to that described above. The devices according to the invention are provided for leisure activities or special applications for humans and animals, such as dogs and horses, in hot areas.

Summary of the Features of the Portable Cooling Device for Humans and Animals According to the Invention

1. The new cooling principle of this device is based on the particularly fine atomization of the continuously supplied water in the cooling region, where it is instantaneously evaporated and thus continually extracts heat from the immediate environment. Ultrasonic water atomizers are particularly suitable for this purpose.

2. The removal of the resulting water vapor takes place by ventilating the cooling region with ambient air and remains limited to this objective in order to ensure optimal effectiveness of cooling.

3. In addition to the upper body, the cooling of the head has also been taken into consideration in the cooling device, because the release of the excess heat takes place principally in these two regions.

4. For this purpose, the cooling device comprises the water atomizer together with reservoir and fan, attached at the bottom to the back of the body and additionally in the head region, wherein the power and thus also the cooling intensity thereof can be chosen as required by means of changing the voltage.

5. The surface of the receptacle, vest or cover facing the back of the body is specially structured and in this connection is blackened, in order to facilitate sufficient ventilation and optimal heat exchange and as a result to ensure the highest possible cooling.

6. A heat-insulating cover of the upper body together with a cooling device, which has a heat-insulating function reflecting and scattering thermal and light radiation, serves for protection against heat from outside. After being turned inside out, as required, the cover can also serve as a good protection against cold.

7. The electrical cooling components can be combined into a unit and thus can be more easily stowed, handled or replaced.

8. Alternatively, the electrical components can be fixed by means of magnets to the modified belt and can be electrically connected to a battery without problems. This cooling device reduced to a minimum is nevertheless sufficiently powerful and, moreover, can be used in practically any environment.

9. An important advantage of the cooling device is the autonomous power supply in sunny areas.

Initial Technical Data

d.c. voltage 5 V or 12 V

current consumption up to 10 W/h

water consumption 50-150 ml/h

required solar surface approx. 15 dqm (30×50 cm)

maximum size of the combined cooling device: 40×50×5 cm

weight: up to 1.2 kg

### LIST OF REFERENCE SIGNS

1 receptacle

2 cooling module, module

2.1 first fan

2.2 first water atomizer

2.3 battery

2.4 voltage controller

3 framework

4 sunshade

5 second water atomizer together with reservoir

6 second fan

7 solar panel

8 thermal protection cover, cover

9 structured surface

10 portable cooling device

11 ventilation and cooling region

12 ventilation and cooling region

13 surface to be cooled, back

14 surface to be cooled, head

1. A portable cooling device in the form of a backpack-like receptacle (1) for humans or vest or blanket for animals,
wherein the portable cooling device (10) has at least two water atomizers together with reservoir (2.2, 5), and at least two fans (2.1, 6),
wherein, in use, a ventilation and cooling region (11, 12) is formed between the portable cooling device (10) and the surface (13, 14) of the human or animal to be cooled and
wherein the fan (2.1, 6) and the water atomizer together with reservoir (2.2, 5) are, in particular, arranged adjacent to one another for ventilating and cooling this ventilation and cooling region (11, 12).

2. The portable cooling device according to claim 1,
wherein the portable cooling device (10) worn on the back has a structured surface so that the ventilation and cooling region is formed between the receptacle and the back of the body.

3. The portable cooling device according to claim 1,
wherein the receptacle surface facing the back is structured in such a way that optimized conditions are ensured for heat exchange and for ventilation between the receptacle and the back of the body during the use.

4. The portable cooling device for animals according to claim 1,
wherein the cooling device is constructed as a vest or blanket and that wherein the inner side of the vest or blanket is structured so that the ventilation and cooling region is formed between the vest or the blanket and the back of the body.

5. The portable cooling device according to claim 1,
wherein the ambient air used for the ventilation and the atomized water required for cooling are controllable in terms of quantity as required.

6. The portable cooling device according to claim 1,
wherein the device is equipped with a battery for the required electrical energy for ventilation and for atomization of water and as an intermediate store for the solar energy.

7. The portable cooling device according to claim 1,
wherein as a simplified cooling device, instead of a backpack-like receptacle, a belt which has two rows of magnetized rivets, each row being electrically connected with the same polarity, and has a connection for the battery, serves as substrate and electrical connection simultaneously for the fan and water atomizer to the battery.

8. The portable cooling device according to claim 1,
wherein as a simplified cooling device the fans and water atomizers are equipped with permanent magnets in pairs and are electrically connected with the same polarity to the battery connection on the belt and thus are fixed, so that on each perpendicular pair of belt rivets they ensure electrically correct connection to the battery and are retained magnetically.

9. The portable cooling device according to claim 1,
wherein it has a solar panel which can be oriented horizontally and/or according to the position of the sun overhead.

10. The portable cooling device according to claim 1,
wherein it has a heat-insulating cover which reflects thermal radiation and scatters light for humans and for the cooling device.

11. The portable cooling device according to claim 1,
wherein the cooling effect principally takes place by use of water atomizers with the formation of very fine drops of water with an average size below 20 μm.

12. The portable cooling device according to claim 11,
wherein the atomization of water preferably takes place using ultrasound.

13. The portable cooling device according to claim
wherein the water used in the water atomizers contains no mineral and organic components.

14. The portable cooling device according to claim 13,
wherein hydrogen peroxide is added to the water used in the water atomizers.

15. The portable cooling device according to claim 14,
wherein the concentration of hydrogen peroxide in the water is in the range from 0.05 to 0.5% by weight.
